(19) United States
(12) Patent Application Publication
(10) Pub. No.: US 2009/0062722 A1
(43) Pub. Date: Mar. 5, 2009

(54) Drug sheet

(76) Inventors: Wataru Watanabe, Usuki (JP); Yuichiro Asano, Tokyo (JP); Yozo Niina, Usuki (JP)

Correspondence Address: WENDEROTH, LIND & PONACK, L.L.P., 2033 K STREET N. W., SUITE 800, WASHINGTON, DC, 20006-1021, US

(21) Appl. No.: 12/222,994

(22) Filed: Aug. 21, 2008

(30) Foreign Application Priority Data
Sep. 5, 2007 (JP) 2007-229978

(51) Int. Cl. A61N 1/30; A61L 15/00

(52) U.S. Cl. 604/20; 604/304

(57) ABSTRACT

A drug sheet includes a base fabric carrying a therapeutic agent containing an electrolytic drug component and applied to or impregnated into a skin-abutting surface of the base fabric. Small needle members carrying small needle-shaped protrusions are scattered on the skin-abutting surface so that the needle-shaped protrusions protrude from the skin-abutting surface. The needle members include a first group of needle members made of aluminum and provided on a first area of the skin-abutting surface, and a second group of needle members made of silver and provided on a second area of the skin-abutting surface. Due to a difference in standard electrode potential between the first and second groups of needle members, electric current flows between the first and second groups of needle members through skin when the needle-shaped protrusions are stuck in the skin. This improves the absorption rate of the drug component.

### BACKGROUND OF THE INVENTION

The present invention relates to a wet type drug sheet which carries a therapeutic agent applied to or impregnated into its skin-abutting surface.

Such drug sheets are widely used as convenient and simple means for treating e.g. neuralgia and stiff shoulders by allowing the therapeutic agent carried on the sheet to be absorbed into the body through the skin. But it is known that only about 20 to 30% of the therapeutic agent carried on such conventional drug sheets are absorbed into the body, so that it is desired to increase the absorption rate of the therapeutic agent.

One of the inventors of the present application proposed (in JP Patent Publication 3-69545B) an electric therapeutic device which ionizes drug components by passing electric current through the skin, thereby improving the penetration of the drug components. This device comprises a small flat battery, a sheath enclosing one of the electrode surfaces and the side of the battery with a gap left between the sheath and the battery, an insulating member insulating the electrodes from each other, an electrically conductive fluid therapeutic agent filling the gap, and a peelable sheet closing the opening of the gap. When in use, the sheet is peeled off to expose the electrode surface, and the electrode surface is brought into contact with the skin. He also proposed (in JP Patent Publication 2005-192848A) an electric therapeutic device which is a battery comprising an anode plate adapted to be stuck on the skin-abutting surface of a drug sheet carrying an ionizable electrolytic therapeutic agent applied to or impregnated into the skin-abutting surface, a cathode plate adapted to be brought into abutment with skin, an electrolyte disposed between the anode and cathode plates, and an insulating annular seal member sealing the outer periphery of the gap between the anode and cathode plates. Simply by sticking this device on the drug sheet, it is possible to dramatically increase the absorption rate of the drug components carried on the drug sheet.

Another inventor of the present application proposed (in JP Patent Publication 2006-158579A) an electric therapeutic device which comprises an anode plate adapted to be stuck on the skin-abutting surface of a drug sheet carrying a therapeutic agent applied to or impregnated into the skin-abutting surface, a cathode plate adapted to be brought into abutment with skin, a battery disposed between the anode and cathode plates with its anode and cathode kept in contact with the anode and cathode plates, respectively, and an insulating annular seal member sealing the outer periphery of the gap between the anode and cathode plates. The anode plate has an extension radially outwardly protruding from the annular seal member and thus adapted to be brought into contact with skin. With this arrangement, since electric current flows between the anode and cathode plates through skin, it is possible to increase the absorption rate of the drug components of the therapeutic agent even if the drug components are not ionizable.

Because the electric therapeutic device disclosed in either of the latter two publications includes a battery, it is possible to increase the absorption rate of the drug components simply by sticking it on the skin-abutting surface. But because such a therapeutic device includes a battery, it is necessary to separately dispose of the battery and other elements after use. Also, such a therapeutic device adds to the cost of the drug sheet.

An object of the present invention is to increase the absorption rate of the drug components carried on a wet type drug sheet without using a separate battery.

### SUMMARY OF THE INVENTION

In order to achieve this object, the present invention provides a wet type drug sheet comprising a base sheet member having a skin-abutting surface configured to be brought into contact with skin, a therapeutic agent applied to or impregnated into the skin-abutting surface, and a plurality of small needle members each carrying at least one small needle-shaped protrusion and scattered on the skin-abutting surface with the needle-shaped protrusions protruding from the skin-abutting surface.

With this arrangement, by sticking the needle-shaped protrusions of the respective needle members into skin, the drug component is injected under the skin by flowing along the needle-shaped protrusions. Thus, it is possible to increase the absorption rate of the drug component carried on the wet type drug sheet without using a separate battery. The needle members may be made of a metal, a resin or a ceramic material.

The needle-shaped protrusions protrude from the skin-abutting surface to a height of 10 to 150 μm, preferably 20 to 120 μm. Human skin comprises, from the surface, the cornified layer, the epidermis, which comprise live cells, and the dermis, where there are capillary vessels and nerves. The cornified layer is 10-15 μm thick, and the dermis is 50-100 μm thick. Since the needle-shaped protrusions protrude to a height of not less than 10 μm, their tips reach the epidermis, so that it is possible to deliver the drug components to the live cells forming the epidermis. For this reason, the needle-shaped protrusions preferably protrude to a height of not less than 20 μm. The needle-shaped protrusions should not protrude to a height of more than 150 μm to prevent infection through capillary vessels in the dermis. For this reason, the needle-shaped protrusions preferably protrude to a height of not more than 120 μm.

Preferably, the small needle members each comprise first three needle-shaped protrusions and second three needle-shaped protrusions, the first three protrusions extending from the center of the needle member in three axial directions intersecting each other at right angles, respectively, the second three protrusions extending from the center of the needle member in opposite directions to the respective first three protrusions. With this arrangement, simply by randomly scattering the needle members on the skin-abutting surface, at least one of the six protrusions of each needle member protrudes from the skin-abutting surface to a sufficient height.

By providing a mesh for holding the needle members in position on the skin-abutting surface, it is possible to prevent separation of the needle members from the skin-abutting surface.

If the therapeutic agent contains an electrolytic drug component, a first group of the needle members and a second group of the needle members may be made of two different kinds of metals having different standard electrode potentials from each other, and scattered on the skin-abutting surface so as to be spaced from each other. Due to the difference in electric potential between the first and second groups of the needle members, electric current flows between these two groups of the needle members through skin, which further increases the absorption rate of the drug component.

Preferably, the first and second groups of the needle members are provided on first and second areas of the skin-abutting surface, respectively, which are spaced from each other. With this arrangement, it is possible to reliably prevent the two groups of needle members from coming into contact with each other.

If the therapeutic agent contains an electrolytic drug component, the drug sheet may further comprise an electrode plate stuck on the skin-abutting surface and made of or plated with a first metal, and at least a part of the needle members may be made of or plated with a second metal having a different standard electrode potential from the first metal, and spaced from the electrode plate. With this arrangement, too, due to the difference in electric potential between the electrode plate and the needle members, electric current flows between the electrode plate and the needle members through skin, which further increases the absorption rate of the drug component.

If the therapeutic agent contains an electrolytic drug component, the drug sheet may further comprise two electrode plates stuck on the skin-abutting surface so as to be spaced from each other, the electrode plates being made of two different kinds of metals having different standard electrode potentials from each other. With this arrangement, too, due to the difference in electric potential between the electrode plates, electric current flows between the electrode plates through skin, which further increases the absorption rate of the drug component.

By providing a non-electrically conductive mesh holding the needle members in position, it is possible to prevent short-circuiting between the abovementioned first and second groups of the needle members, between the electrode plate and the needle members, or between the abovementioned two electrode plates, and also prevent separation of the needle members from the skin-abutting surface.

If the above-mentioned two electrode plates are stuck on the skin-abutting surface, by providing an electrically conductive mesh holding the needle members in position so as to be spaced from at least one of the two electrode plates, it is possible to prevent short-circuiting between the two electrodes, and also prevent separation of the needle members from the skin-abutting surface.

### BRIEF DESCRIPTION OF THE DRAWINGS

Other features and objects of the present invention will become apparent from the following description made with reference to the accompanying drawings, in which:

FIG. 1A is a plan view of a drug sheet according to a first embodiment of the present invention;

FIG. 1B is a vertical sectional side view of FIG. 1A;

FIG. 2A is an enlarged perspective view of a small needle member shown in FIGS. 1A and 1B;

FIG. 2B is a partial vertical sectional side view of the drug sheet of the first embodiment, showing the position of a needle member relative to the skin-abutting surface;

FIG. 3A is a plan view of a drug sheet according to a second embodiment of the present invention;

FIG. 3B is a vertical sectional side view of FIG. 3A;

FIG. 4A is an enlarged perspective view of a small needle member shown in FIGS. 3A and 3B;

FIG. 4B is a partial vertical sectional side view of the drug sheet of the first embodiment, showing how the needle member of FIG. 4A is held in position on the skin-abutting surface;

FIG. 5A is a plan view of a drug sheet according to a third embodiment of the present invention;

FIG. 5B is a vertical sectional side view of FIG. 5A;

FIG. 6A is a plan view of a drug sheet according to a fourth embodiment of the present invention;

FIG. 6B is a vertical sectional side view of FIG. 6A;

FIG. 7A is a plan view of a drug sheet according to a fifth embodiment of the present invention;

FIG. 7B is a vertical sectional side view of FIG. 7A;

FIG. 8A is a plan view of a drug sheet according to a sixth embodiment of the present invention; and

FIG. 8B is a vertical sectional side view of FIG. 8A.

### DETAILED DESCRIPTION OF THE PREFERRED EMBODIMENTS

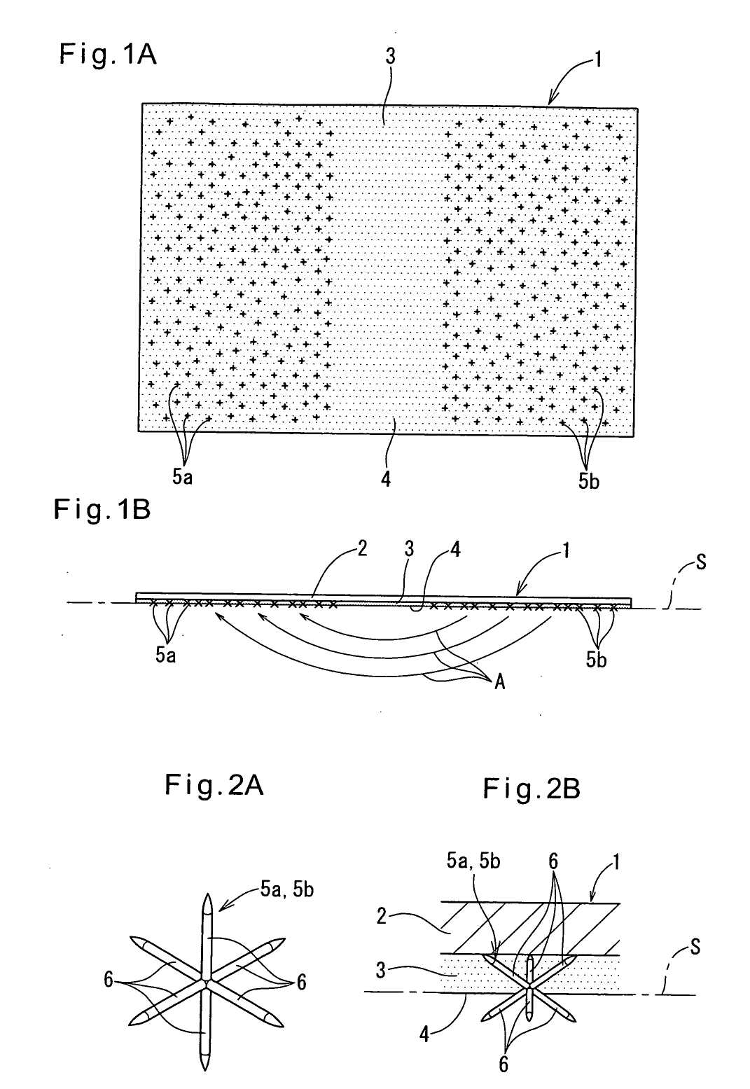

Now the embodiments are described with reference to the drawings. The drug sheet 1 according to the first embodiment, shown in FIGS. 1A, 1B, 2A and 2B, is a wet type sheet comprising, as shown in FIGS. 1A and 1B, a base fabric 2 having a skin-abutting surface 4 to be brought into contact with skin S such as human skin, and a therapeutic agent 3 containing electrolytic drug components and applied to or impregnated into the skin-abutting surface 4. On one of first and second separate areas (left-hand and right-hand sides in FIG. 1A) of the skin-abutting surface 4, a plurality of small needle members 5a made of aluminum and each comprising a plurality of needle-shaped protrusions 6 are scattered. On the other of the first and second areas of the skin-abutting surface 4, a plurality of small needle members 5b made of silver and each comprising a plurality of needle-shaped protrusions 6 are scattered.

FIG. 2A shows an enlarged such small needle member 5a (5b). As shown, each small needle member 5a (5b) comprises first three needle-shaped protrusions 6 extending, from the center of the small needle member, in three axial directions intersecting each other at right angles, and second three needle-shaped protrusions 6 extending from the center of the small needle member, in the directions opposite to the first three needle-shaped protrusions 6, respectively. The needle members thus have the shape of a three-dimensional cross. Each needle-shaped protrusion 6 is about 100 μm long. As shown in FIG. 2B, most of the small needle members 5a (5b) have three of the six needle-shaped protrusions 6 supported by the base fabric 2 so that the other three protrusions 6 protrude from the skin-abutting surface 4 to a height of about 40 to 80 μm. Thus, when the sheet 1 is applied to the skin S, these other three protrusions 6 of each needle member 5a (5b) penetrate into the skin S, allowing the drug components of the therapeutic agent 3 to be injected under the skin by flowing along these needle-shaped protrusions 6.

The standard electrode potential of the small needle members 5a, which are made of aluminum, is −1.66 V, while the standard electrode potential of the small needle members 5b, which are made of silver, is +0.80 V. Thus, as shown in FIG. 1B, electric current A flows from the small needle members 5b, which are made of silver and has a high standard electrode potential, through the skin S, to the small needle members 5a, which are made of aluminum and has a low standard electrode potential. This ionizes the drug components which are injected along the needle-shaped protrusions 6 and those absorbed directly from the skin-abutting surface 4, thus improving the absorption rate of the drug components.

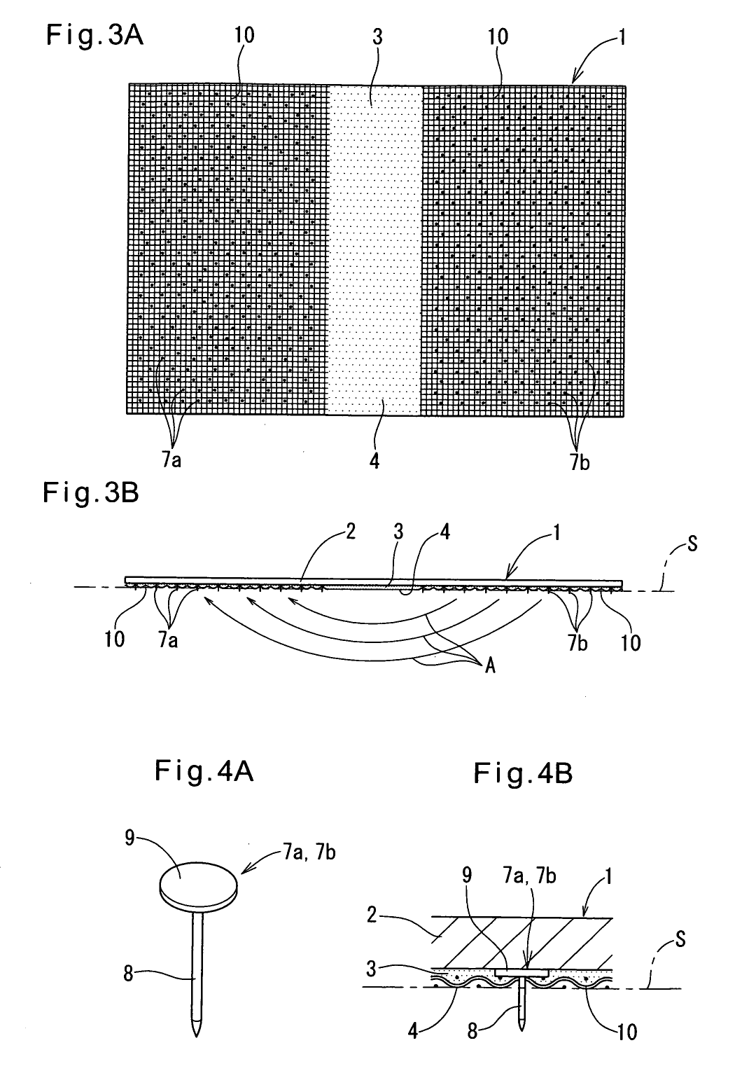

FIGS. 3A, 3B, 4A and 4B show the second embodiment. The drug sheet of this embodiment is basically of the same structure as the drug sheet of the first embodiment. That is, as shown in FIGS. 3A and 3B, on one and the other of the left-hand and right-hand separate areas of the skin-abutting surface 4, a plurality of small needle members 7a and 7b made of aluminum and silver, respectively, are scattered. But this embodiment differs from the first embodiment in that, as shown enlarged in FIG. 4A, each needle member 7a (7b) is in the shape of a tack comprising a disk-shaped head 9 and a needle-shaped protrusion 8 extending from the head 9 at a right angle, and that the needle members 7a and 7b are held in position on the skin-abutting surface 4 by means of a non-electrically conductive mesh 10 made of a resin. The needle-shaped protrusions 8 of the needle members 7a and 7b are about 130 μm long.

As shown in FIG. 4B, each small needle member 7a (7b) has its head 9 held on the base fabric 2 by means of the mesh 10 with the needle-shaped protrusion 8 penetrating through the mesh 10 and protruding from the skin-abutting surface 4 to a height of about 50 to 80 μm. In this embodiment too, as shown in FIG. 3B, electric current A flows from the small needle members 7b, which are made of silver, to the small needle members 7a, which are made of aluminum, thus improving the absorption rate of the drug components injected along the needle-shaped protrusions 8 and those absorbed directly from the skin-abutting surface 4.

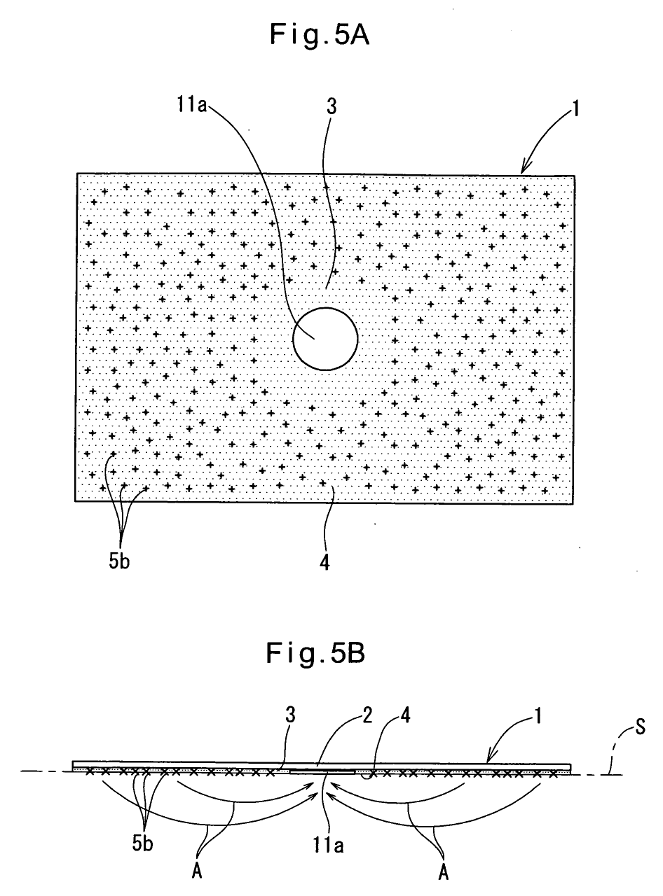

FIGS. 5A and 5B show the third embodiment. The drug sheet 1 of this embodiment includes an aluminum electrode plate 11a stuck on the skin-abutting surface 4 at its central portion. Three-dimensional cross-shaped small needle members 5b made of silver, i.e. needle members 5b used in the first embodiment, are scattered around the electrode plate 11a so as to be spaced from the electrode plate 11a.

In this embodiment, as shown in FIG. 5B, electric current A flows from the small needle members 5b, which are made of silver and thus have a high standard electrode potential, through the skin S, to the aluminum electrode plate 11a, which has a low standard electrode potential, thus improving the absorption rate of the drug components injected along the needle-shaped protrusions 6 and those absorbed directly from the skin-abutting surface 4.

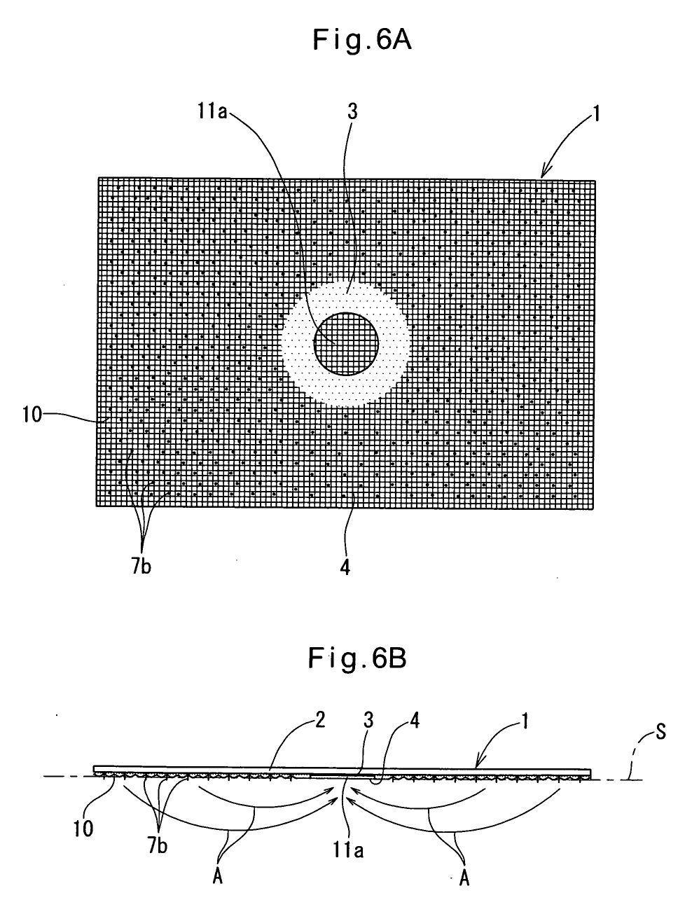

FIGS. 6A and 6B show the fourth embodiment. The drug sheet 1 of this embodiment is basically of the same structure as the drug sheet of the third embodiment. That is, a plurality of small needle members made of silver are scattered around an aluminum electrode plate 11a stuck on the skin-abutting surface 4 at its central portion so as to be spaced from the electrode plate 11a. This embodiment differs from the third embodiment in that the small needle members are the tack-shaped needle members 7b used in the second embodiment, and that these small needle members 7b are held in position by a non-electrically conductive mesh 10 made of a resin. In this embodiment too, as shown in FIG. 6B, electric current A flows from the small silver needle members to the aluminum electrode plate 11a.

In the third and fourth embodiments, the electrode plate 11a is made of aluminum, which has a low standard electrode potential, and the small needle members 5b, 7b are made of silver, which has a high standard electrode potential, so that electric current A flows from the small needle members 5b, 7b to the aluminum electrode plate 11a. But conversely, the electrode plate may be made of silver, which has a high standard electrode potential, and the small needle members may be made of aluminum, which has a low standard electrode potential, so that electric current A flows from the electrode plate to the small needle members.

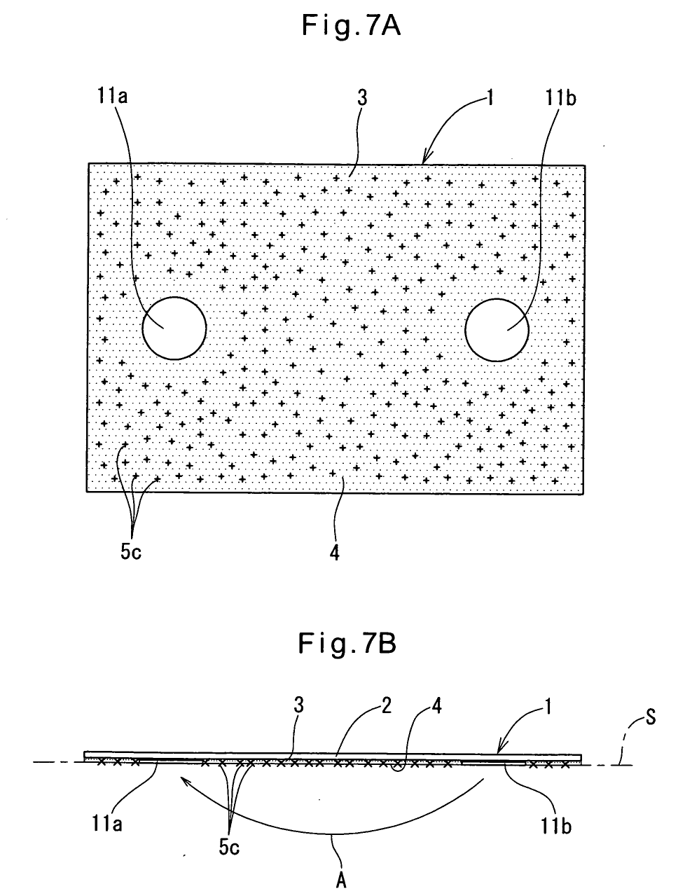

FIGS. 7A and 7B show the fifth embodiment. The drug sheet 1 of this embodiment includes electrode plates 11a and 11b made of aluminum and silver, respectively, and provided on the skin-abutting surface 4 at its respective end portions so as to be spaced from each other. Three-dimensional cross-shaped small needle members 5c similar in shape to those used in the first embodiment but made of a resin or a ceramic material are scattered on the skin-abutting surface 4 where there are no electrode plates 11a and 11b.

In this embodiment, the drug components of the therapeutic agent 3 are injected under the skin by flowing along the needle shaped protrusions 6 of the small needle members 5c that are stuck in the skin S. Also, as shown in FIG. 7B, electric current A flows from the electrode plate 11b, which is made of silver and thus has a high standard electrode potential, through the skin, to the electrode plate 11a, which is made of aluminum and thus has a low standard electrode potential, thus improving the absorption rate of the drug components injected along the needle-shaped protrusions 6 and those absorbed directly from the skin-abutting surface 4.

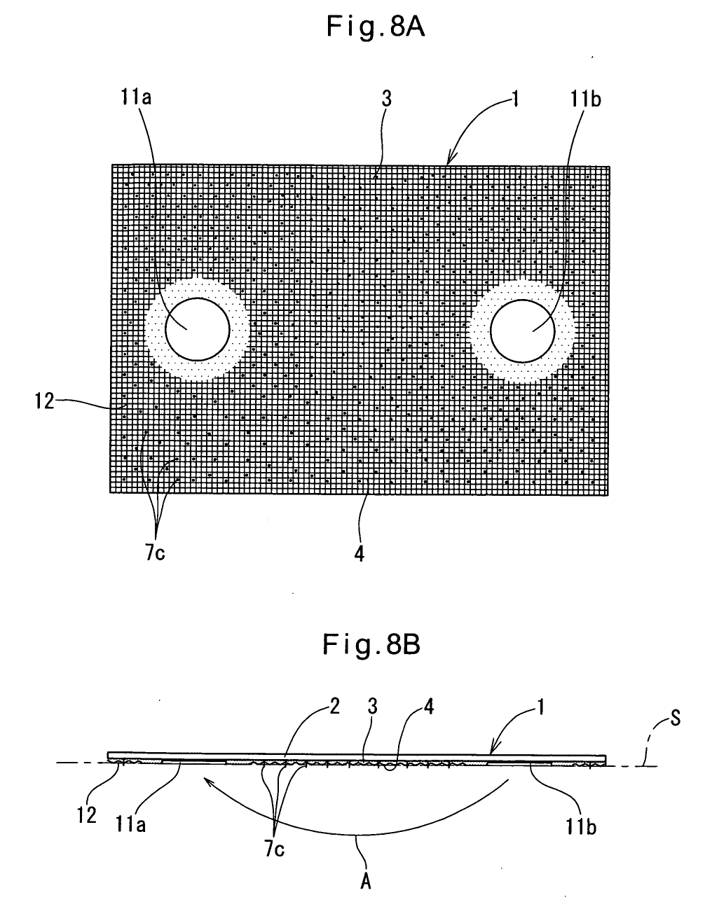

FIGS. 8A and 8B show the sixth embodiment. The drug sheet 1 of this embodiment is basically of the same structure as the drug sheet of the fifth embodiment. That is, small needle members made of a resin or a ceramic material are scattered on the skin-abutting surface 4 where there are no electrode plates 11a and 11b. This embodiment differs from the fifth embodiment in that the small needle members are tack-shaped needle members 7c similar to those used in the second embodiment, and that these small needle members 7c are held in position by an electrically conductive mesh 12 made of a metal. The electrically conductive mesh 12 is kept out of contact with the electrode plates 11a and 11b. In this embodiment too, as shown in FIG. 8B, electric current A flows from the silver electrode plate 11b to the aluminum electrode plate 11a.

In the above embodiments, as two different metals having different standard electrode potentials, aluminum and silver are used for the respective two groups of small needle members, for the needle members and the single electrode plate, or for the two electrode plates. But such two different metals are not limited to aluminum and silver. For example, such two different metals may be magnesium and gold. Also, instead of forming the entire small needle members and the electrode plate or plates from such metals, some or all of the needle members and/or the electrode plate or plates may be plated with one or both of such metals.

In the embodiments, three-dimensional cross-shaped needle members and tack-shaped needle members are shown. But the small needle members of the present invention may have a different shape, provided they each have at least one needle-shaped protrusion which stably and reliably protrudes from the skin-abutting surface of the drug sheet.

In the embodiments, the therapeutic agent contains an electrolytic drug component. But the drug sheet according to the present invention is applicable to the case in which a non-electrolytic therapeutic agent is used. In this case, it is possible to increase the absorption rate of the drug component only by the effect of injection of the drug component along the needle-shaped protrusions stuck in the skin.

What is claimed is:

1. A wet type drug sheet comprising a base sheet member having a skin-abutting surface configured to be brought into contact with skin, a therapeutic agent applied to or impregnated into said skin-abutting surface, and a plurality of small needle members each carrying at least one small needle-shaped protrusion and scattered on the skin-abutting surface with said needle-shaped protrusions protruding from the skin-abutting surface.

2. The drug sheet of claim 1 wherein said needle-shaped protrusions protrude from the skin-abutting surface to a height of 10 to 150 μm.

3. The drug sheet of claim 1 wherein each of said small needle members comprise first three needle-shaped protrusions and second three needle-shaped protrusions, said first three protrusions extending from a center of the needle member in three axial directions intersecting each other at right angles, respectively, said second three protrusions extending from the center of the needle member in opposite directions to the respective first three protrusions.

4. The drug sheet of claim 1 further comprising a mesh holding said needle members in position on the skin-abutting surface.

5. The drug sheet of claim 1 wherein said therapeutic agent contains an electrolytic drug component, wherein a first group of said needle members are made of or plated with a first metal and a second group of said needle members are made of or plated with a second metal having a higher standard electrode potential than said first metal, and wherein said first group of said needle members and said second group of said needle members are scattered on the skin-abutting surface so as to be spaced from each other.

6. The drug sheet of claim 5 wherein said first group of said needle members and said second group of said needle members are provided on first and second areas of the skin-abutting surface, respectively, which are spaced from each other.

7. The drug sheet of claim 1 wherein said therapeutic agent contains an electrolytic drug component, said drug sheet further comprising an electrode plate stuck on said skin-abutting surface and made of or plated with a first metal, and wherein at least a part of said needle members are made of or plated with a second metal having a different standard electrode potential from said first metal, and spaced from said electrode plate.

8. The drug sheet of claim 1 wherein said therapeutic agent contains an electrolytic drug component, said drug sheet further comprising two electrode plates stuck on said skin-abutting surface so as to be spaced from each other, said electrode plates being made of two different kinds of metals having different standard electrode potentials from each other.

9. The drug sheet of claim 5 further comprising a non-electrically conductive mesh holding said needle members in position.

10. The drug sheet of claim 8 further comprising an electrically conductive mesh holding said needle members in position and spaced from at least one of said two electrode plates.
